(19) United States
(12) Patent Application Publication
(10) Pub. No.: US 2010/0278875 A1
(43) Pub. Date: Nov. 4, 2010

(54) PROSTAGLANDIN E2 (PGE2) AS AN ADJUVANT IN MONOCLONAL ANTIBODY GENERATION

(76) Inventors: Jill Giles-Komar, Downingtown, PA (US); Michael A. Rycyzyn, Berwyn, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/376,485

(22) PCT Filed: Sep. 28, 2007
(86) PCT No.: PCT/US07/79968

§ 371 Date: Feb. 5, 2009

Related U.S. Application Data
(60) Provisional application No. 60/827,229, filed on Sep. 28, 2006.

(51) Int. Cl. A61K 31/201; C07K 16/00; G01N 33/53; C12P 21/00; A61P 37/04

(52) U.S. Cl. 424/283.1; 530/387.1; 435/7.2; 435/70.2

(57) ABSTRACT

The present invention provides PGE2 as a novel adjuvant for enhancing the immune response in a host, as well as methods of using PGE2 to enhance B cell response and thereby increasing antibody titer against a given immunogen are also disclosed and antibodies produced by at least one method of the present invention.

### BACKGROUND OF THE INVENTION

1. Field of the Invention

The present invention relates to methods of using PGE2 as an adjuvant for enhancing the immune response in a host, in order to aid in production of antibodies.

2. Related Art

The use of monoclonal antibodies (mAbs) as therapeutic reagent has become an effective approach for the treatment of various diseases. In addition, mAbs are powerful tools to gain a better understanding of the immuno-pathogenesis of various diseases. A standard method for generating mAbs consists of fusing myeloma cells with lymph node cells or splenocytes harvested from immunized Balb/c mice. Balb/c mice represent the host of choice for raising mAbs because they are readily available. More importantly, the immune response in Balb/c mice sensitized with foreign T-dependent antigens is characterized by a polarization of their T-cell derived cytokine production toward a Th2-like phenotype. This Th2-like response is accompanied by the generation of high levels of antigen-specific Abs, which correlates with an increase in the frequency of antigen-specific B cell clones and an increase in the number of hybrids following B cell fusion to obtain mAbs.

However, many immunogens are not capable of triggering an adequate antibody response in the mice. This means that there are only few B cells producing antibody against the immunogen, making it difficult to isolate these cell lines after forming hybridomas. The low antibody response results because the immunogens do not elicit adequate T cell help to expand B cell clones specific for the immunogen to an appreciable extent. In addition, the generation of mAbs against some immunogens prove difficult due to toxicity issues following repeated injections.

In order to increase the number of B cells which produce antibody against an immunogen, one often uses adjuvants which cause an enhanced antibody response against the immunogen. Adjuvants are compounds which, when administered with an immunogen, enhance the immune systems response to produce higher antibody titers and prolonged host response. Commonly used adjuvants include Incomplete Freund's Adjuvant, which consists of a water in oil emulsion, Freund's Complete Adjuvant, which comprises the components of Incomplete Freund's Adjuvant, with the addition of Mycobacterium tuberculosis, and alum. However, regulatory agencies discourage the use of certain adjuvants due to their serious side effects. Moreover, while these adjuvants can boost the humoral response against foreign immunogens, they also denature some protein immunogens. This can affect the processing and presentation of key immunogenic epitopes for the generation of bioreactive antibodies. Therefore, there is a need to provide new adjuvants that overcome one more of these problems.

### SUMMARY OF THE INVENTION

In one aspect, the present invention provides PGE2 as a novel adjuvant for enhancing immune response in a host. In one embodiment, the present invention provides PGE2 as an adjuvant for enhancing B cell response in the animal.

In another aspect, the present invention provides a method to enhance immune response against a given immunogen in a host. In one embodiment, the method comprises administering to the host the immunogen of interest and an effective adjuvanting amount of PGE2. The immune response enhanced by the method of the present invention may be B cell response and may be exemplified by increased antibody titers.

In another aspect, the present invention provides an improved method for producing antibodies against an immunogen, the method comprising administering an immunogen and an effective adjuvanting amount of PGE2, thereby increasing the immune response against the immunogen, and screening for antibodies, or cells producing antibodies, which are specifically reactive with the immunogen. The method of the present invention provides a more efficient way of generating antibodies. Accordingly, in another aspect, the present invention provides antibodies produced using the improved method of the present invention. The antibodies produced using the present invention can be used for therapeutic, diagnostic, and/or research purposes.

### DESCRIPTION OF THE INVENTION

All publications or patents cited herein are entirely incorporated herein by reference as they show the state of the art at the time of the present invention and/or to provide description and enablement of the present invention.

The present invention provides PGE2 as a novel adjuvant, which can be effectively used for enhancing immune response in a host. The immune response enhanced may be B cell response. In one embodiment, the present invention provides PGE2 as an adjuvant to increase antibody titers against a given immunogen in mice.

PGE2 is an arachidonic acid (AA) metabolite produced by various types of cells. It regulates a broad range of physiological activities in the endocrine, cardiovascular, gastrointestinal, neural, reproductive, and immune systems, and maintains the local homeostasis. PGE2 synthesis occurs in three steps. First, AA is released from membrane phospholipids via the action of phospholipase A2. Next, AA is converted to PGG2 and then PGH2 by the cyclooxygenases 1 and 2 (Cox-1 and Cox-2). Finally, PGH2 is isomerized to PGE2 by terminal PGE synthase.

In the immune system, PGE2 is mainly produced by APCs such as monocytes, macrophages and dendritic cells. PGE2 are suppressive on Th1-related immune responses. It suppresses IL-2 and IFN-y production by Th1 clones, but not IL-4 and IL-5 production by Th2 clones. In the differentiation phase of naïve T cells, PGE2 inhibits the differentiation of Th1 and IL-12R expression via cAMP accumulation. PGE2 suppresses LPS-induced IL-12 production by APCs, but enhances IL-10 production. In B cells, PGE2 enhances IgE production by IL-4 and LPS-stimulated B cells in vitro. It is now discovered that PGE2 as a key player in the generation of a Th2 response.

The term “immunogen” as used herein means any molecule that can potentially elicit an immune response in a subject. Since some immunogens do not elicit an immune response when administered in the absence of an adjuvant, the term “immunogen” encompasses molecules that only elicit an immune response when co-administered with an adjuvant.

The term “adjuvant” as used herein refers to a substance which enhances the immune-stimulating properties of an immunogen. Adjuvants have the capacity of influencing antibody titer, response duration, isotype, avidity, and other properties of immunity. The use of adjuvants is preferred or required for many immunogens which by themselves are weakly immunogenic. Adjuvants may act through a number of different mechanisms. Presently known and/or utilized adjuvants are limited by toxic and allergenic effects, or are extremely expensive to produce.

As used herein, the term “enhancing” or “enhanced” regarding the immune response to an immunogen describes increasing, strengthening or inducing an immune response to the immunogen. In the present invention, PGE2, as an adjuvant, enhances immune responses not only to strong immunogens, but also to difficult/nominal immunogens.

As used herein, the term “antibody” includes polyclonal antibodies and monoclonal antibodies. In general, antibodies are proteins or polypeptides that exhibit binding specificity to a specific immunogen. Intact antibodies are heterotetrameric glycoproteins, composed of two identical light chains and two identical heavy chains. Typically, each light chain is linked to a heavy chain by one covalent disulfide bond, while the number of disulfide linkages varies between the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa (κ) and lambda (λ), based on the amino acid sequences of their constant domains. Immunoglobulins can be assigned to five major classes, namely IgA, IgD, IgE, IgG and IgM, depending on the heavy chain constant domain amino acid sequence. IgA and IgG are further sub-classified as the isotypes IgA1, IgA2, IgG1, IgG2, IgG3 and IgG4.

The term “monoclonal antibody” as used herein refers to a preparation of antibody molecules of single molecular composition. A monoclonal antibody displays a single binding specificity and affinity for a particular epitope. Monoclonal antibodies include murine, human, humanized and chimeric monoclonal antibodies.

The present invention also provides a method to enhance immune response against a given immunogen in a host. In one embodiment, the method comprises administering to the host the immunogen of interest and an effective adjuvanting amount of PGE2. The immune response enhanced by the method of the present invention may be B cell response and may be exemplified by increased antibody titers.

As used herein, the term “effective adjuvanting amount” refers to the amount of an adjuvant, when administered simultaneously or sequentially with an immunogen, produces enhancement of the effect obtained with the immunogen alone or alternatively induces an immune response to the immunogen. One skilled in the art is expected to be able to readily determine suitable amounts of PGE2 to adjuvant certain immunogens. Such amounts will typically depend upon the nature of the immunogen, the dosage amounts of the immunogen, the species and physical conditions of the host, as well as the route of administration. For example, an effective adjuvanting amount of PGE2 described herein can range, from about 0.1 nmol to about 10 nmol, such as but not limited to, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 nmol, or any range or value therein, such as but not limited to, 0.01-10 nmol, 0.05-5 nmol, 0.1-2 nmol, 0.5-0.9 nmol, 0.1-1.0, 0.01-0.05, 0.05-0.1, 0.1-0.5, 0.6-1.0, 1-5, 5-10, 10-20, 20-30 nmol, or any range or value therein.

PGE2 may be administered simultaneously or sequentially with the immunogen. When PGE2 is administered simultaneously with the immunogen, both the immunogen and PGE2 can form a part of the same composition. Alternatively, the adjuvanting effect of PGE2 may be employed by administering PGE2 separately from the immunogen. When administered separately, PGE2 is preferably provided in a suitable carrier, such as saline or PBS. PGE2 may be administered contemporaneously with the immunogen, or alternatively, before or after the immunogen administration. The time interval between the administration of the immunogen and PGE2 depends on the immunogen.

An immunogen is administered according to the immunization schedule for the immunogen. For example, a single administration of the immunogen in an amount sufficient to elicit an effective immune response may be used. Alternatively, other regimes of initial administration of the immunogen followed by one or more boosting may be used. When multiple administrations of the immunogen are desired, PGE2 can be administered with the immunogen either only within the first administration or in all of the scheduled administrations. The administration may be via any suitable route, such as intraperitoneal, intravenous, subcutaneous, intramuscular, intradermal, or through footpad injection.

The present invention further provides an improved method for producing antibodies against an immunogen, the method comprising administering an immunogen of interest and an effective adjuvanting amount of PGE2 and thereby increasing the immune response against the immunogen, and screening for antibodies, or cells producing antibodies, which are specifically reactive with the immunogen.

Citations: All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2006); Sambrook, et al., Molecular Cloning: A Laboratory Manual, 2^{nd} Edition, Cold Spring Harbor, N.Y. (1989); Harlow and Lane, Antibodies, a Laboratory Manual, Cold Spring Harbor, N.Y. (1989); Colligan, et al., eds., Current Protocols in Immunology, John Wiley & Sons, Inc., NY (1994-2006); Colligan et al., Current Protocols in Protein Science, John Wiley & Sons, NY, N.Y., (1997-2006).

In general, means for preparing and characterizing antibodies are well known in the art (e.g., Ausubel, Harlow and Lane, and Colligan, supra. However, certain immunogens are immunologically cryptic and generally do not elicit a satisfactory antibody response when given to a host. The present invention provides an improved method for producing antibodies in which the standard methods can be manipulated to promote an antibody response against an immunogen.

To prepare a polyclonal antibody in accordance with the present invention, a host is administered with an immunogen and an effective adjuvanting amount of PGE2. Antisera is collected from the host. A wide range of animal species can be used for the production of antisera. Typically the animal used for production of anti-antisera is a rabbit, a mouse, a rat, a hamster, a guinea pig or a goat. The production of polyclonal antibodies may be monitored by sampling blood of the immunized animal at various points following immunization. One or more booster injection may also be given. The process of boosting and titering is repeated until a suitable titer is achieved. See, e.g., Colligan, chapter 2, supra, which is entirely incorporated herein by references,

To prepare a monoclonal antibody in accordance with the present invention, a host is administered with an immunogen and an effective adjuvanting amount of PGE2. Typically, rodents such as mice and rats may be used. Following immunization, somatic cells with the potential for producing antibodies, specifically B lymphocytes (B cells), are selected for use in the mAb generating protocol. These cells may be obtained from biopsied spleens, tonsils or lymph nodes, or from a peripheral blood sample.

The antibody-producing B lymphocytes from the immunized animal are then fused with cells of an immortal myeloma cell line. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas). For example, where the immunized animal is a mouse, one may use P3-X63/Ag8, X63-Ag8.653, NS1/1.Ag 41, Sp210-Ag14, FO, NSO/U, MPC-11, MPC11-X45-GTG 1.7 and 5194/5XX0 Bul.

Methods for generating hybrids of antibody-producing spleen or lymph node cells and myeloma cells are well known in the art. Generally, somatic cells are mixed with myeloma cells in a 2:1 proportion, though the proportion may vary from about 20:1 to about 1:1, respectively, in the presence of an agent or agents (chemical or electrical) that promote the fusion of cell membranes. Fusion methods using Sendai virus have been described by Kohler & Milstein (1975; 1976), and those using polyethylene glycol (PEG), such as 37% (v/v) PEG, by Gefter et al. (1977). The use of electrically induced fusion methods is also appropriate (Goding pp. 71-74, 1986).

The population of hybridomas are cultured in selection media and specific hybridomas are selected. Typically, selection of hybridomas is performed by culturing the cells by single-clone dilution in microtiter plates, followed by testing the individual clonal supernatants (after about two to three weeks) for the desired reactivity. The assay may be radioimmunoassays, enzyme immunoassays, cytotoxicity assays, plaque assays, dot immunobinding assays, and the like.

Antibody producing cells can also be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used as known in the art, e.g., see, Colligan, Harlow and Lane, Ausubel, supra, each of which is entirely incorporated herein by reference.

Methods for engineering or humanizing non-human or human antibodies can also be used and are well known in the art. Generally, a humanized or engineered antibody has one or more amino acid residues from a source which is non-human, e.g., but not limited to, mouse, rat, rabbit, non-human primate or other mammal. These human amino acid residues are often referred to as “import” residues, which are typically taken from an “import” variable, constant or other domain of a known human sequence. Known human Ig sequences are well known in the art and can any known sequence. See, e.g., but not limited to, Kabat et al., Sequences of Proteins of Immunological Interest, U.S. Dept. Health (1983) and PCT publication WO 05/33029 and U.S. Ser. No. 10/872,932, filed Jun. 21, 2004, entirely incorporated herein by reference.

Such imported sequences can be used to reduce immunogenicity or reduce, enhance or modify binding, affinity, on-rate, off-rate, avidity, specificity, half-life, or any other suitable characteristic, as known in the art. Generally part or all of the non-human or human CDR sequences are maintained while the non-human sequences of the variable and constant regions are replaced with human or other amino acids. Antibodies can also optionally be humanized with retention of high affinity for the antigen and other favorable biological properties. To achieve this goal, humanized antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766,886, 5,714,352, 6,204,023, 6,180,370, 5,693,762, 5,530,101, 5,585,089, 5,225,539; 4,816,567, PCT/: US98/16280, US96/18978, US91/09630, US91/05939, US94/01234, GB89/01334, GB91/01134, GB92/01755; WO90/14443, WO90/14424, WO90/14430, EP 229246, Colligan, Ausubel, Harlow and Lane, supra, each entirely incorporated herein by reference, included references cited therein.

The antibody can also be optionally generated by immunization of a transgenic animal (e.g., mouse, rat, hamster, non-human primate, and the like) capable of producing a repertoire of human antibodies, as described herein and/or as known in the art. Cells that produce a desired antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

The method of the present invention thus provides a more efficient way of generating antibodies. Accordingly, the present invention also provides antibodies produced using the improved method of the present invention. The antibodies produced using the present invention can be used for therapeutic, diagnostic, and/or research purposes.

Having generally described the invention, the same will be more readily understood by reference to the following examples, which are provided by way of illustration and are not intended as limiting.

### Example 1

### PGE2 as an Adjuvant Significantly Increased Antibody Titers

To generate antibody against ovalbumin (OVA), Balb/c mice were immunized with 25 ug of OVA emulsified in adjuvant. To test the effect of PGE2, the mice were injected with 1 nmol PGE2 intraperitoneally (i.p.) 3 hours prior to immunization, and again at 24 and 48 hours post immunization. For the control group, the mice were injected (i.p.) with an equal volume of PBS 3 hours prior to immunization. The mice were boosted with 25 ug OVA on Day 14 (i.p.) and Day 28 (subcutaneously). Anti-OVA titers were determined on Day 27 and Day 35. As shown in Table 1, treatment of Balb/c mice with PGE2 significantly enhanced anti-OVA titers.

TABLE 1
Anti-OVA titers in Balb/c mice in the presence or absence
of PGE2 (titers expressed as geometric means)

| Immunization | Day 27 Avg. Titer | Day 35 Avg. Titer |
|---|---|---|
| OVA/PBS | ~1:1600 | ~1:20,480 |
| OVA/PGE2 | ~1:128,000 | ~1:809,500 |

To assess the effect of PGE2 on generating antibodies against difficult targets, a nominal immunogen (AgX) known to be difficult to raise an antibody response was used. Briefly, Balb/c mice were immunized following the same schedule outlined above with either 25 ug of OVA or AgX and titered on Day 27 following 2 i.p. injections. As shown in Table 2, PGE2 addition enhanced anti-OVA titers following only 2 immunogen injections. Therefore, PGE2 enhances immune responses in Balb/c mice and may be used to shorten immunization time lines.

More importantly, PGE2 also increased anti-AgX titers by 2-3-fold following 2 injections. Given the difficult nature of this immunogen to generate antibodies against, this is a significant increase. Therefore, PGE2 can be used in Balb/c mice to enhance immune responses not only to strong immunogens, but also to difficult/nominal immunogens.

TABLE 2
Titers in Balb/c mice against a strong and a nominal
immunogen in the presence or absence of PGE2
(titers are expressed as geometric means)

| Immunization | Day 27 Avg. Titer |
|---|---|
| OVA/PBS | ~1:51,200 |
| OVA/PGE2 | ~1:145,000 |
| AgX/PBS | ~1:9,050 |
| AgX/PGE2 | ~1:25,600 |

In summary, the present invention successfully addresses the shortcomings of the presently known and/or utilized adjuvants by providing PGE2 as a novel adjuvant, which is highly efficient, and induce minimal or no adverse side effects.

It will be clear that the invention can be practiced otherwise than as particularly described in the foregoing description and examples. Numerous modifications and variations of the present invention are possible in light of the above teachings and, therefore, are within the scope of the present invention.

### REFERENCES

Fedyk E R, and Phipps R P. 1996. Prostaglandin E₂ receptors of the EP₂ and EP₄ subtypes regulate activation and differentiation of mouse B-lymphocytes to IgE-secreting cells. Proc. Natl. Acad. Sci. 93:10978.
Green L L. 1999. Antibody engineering via genetic engineering of the mouse: XenoMouse strains are a vehicle for the facile generation of therapeutic human monoclonal antibodies. J. Immunol. Meth. 231:11.
Hilkens G M U, Snijders A, Vermeulen H, Meide P H V D, Wierenga E A, and Kapsenberg M L. 1996. Accessory cell derived IL-12 and prostaglandin E₂ determine the IFN-□ level of activated human CD4+ T cells. J. Immunol. 156:1722.
Ivanov A I, and Romansky A A. 2004. Prostaglandin E₂ as a mediator of fever: synthesis and catabolism. Front. Biosci. 9:1977-1993
Katamura K, Shinataku N, Yamauchi Y, Fukui T, Ohshima Y, Mayumi M, and Furusho K. 1995. Prostaglandin E₂ at priming of naïve CD4+ T cells inhibits acquisition of ability to produce IFN-□ and IL-2, but not IL-4 and IL-5. J. Immunol. 155:4604.
Köhler G and Milstein C. 1975. Continuous cultures of fused cells secreting antibody of predefined specificity. Nature 256: 495-497
Köhler G and Milstein C. 1976. Derivation of specific antibody-producing tissue culture and tumor lines by cell fusion. Eur. J. Immunol. 6:511-519.
Kuroda E, Sugiura T, Zeki K, Yoshida Y and Yamishita U. 2000. Sensitivity difference to the suppressive effect of prostaglandin E₂ among mouse strains: a possible mechanism to polarize Th2 type response in Balb/c mice. J. Immunol. 164:2386-2395.
Kuroda E and Yamashita U. 2003. Mechanisms of enhanced macrophage-mediated prostaglandin E₂ production and its suppressive role in Th1 activation in Th2-dominant Balb/c mice. J. Immunol. 170:757-764.
Lonberg N, Taylor L D, Harding F A, Trounstine M et al. 1994. Antigen-specific human antibodies from mice comprising four distinct genetic modifications. Nature 368:856.
Phipps R P, Stein S H, and Roper R L. 1991. A new view of prostaglandin E regulation in the immune response. Immunol. Today 12:349.
Reiner SL and Locksley R M. 1995. The regulation of immunity to Leishmania major. Annu. Rev. Immunol. 13:151-177.
Snijdewint F G M, Kalinski P, Wierenga E A, Bos J D, and Kapsenberg M L. 1993. Prostaglandin E₂ differentially modulate cytokine secretion profiles of human T helper lymphocytes. J. Immunol. 150:5321.
Tineke C T M, Kraan V D P, Boeije L C M, Smeenk R J T, Wijdenes J, and Aarden L A. 1995. Prostaglandin E₂ is a potent inhibitor of human interleukin 12 production. J. Exp. Med. 181:775.
Wu C Y, Wang K, McDyer J F, and Seder R A. 1998. Prostaglandin E₂ and dexamethasone inhibit IL-12 receptor expression and IL-12 responsiveness. J. Immunol. 161:2723.

What is claimed is:

1. A method for enhancing immune response against an immunogen in a host, comprising administering to the host the immunogen and an effective adjuvanting amount of PGE2.

2. The method of claim 1, wherein the immunogen and PGE2 are administered simultaneously.

3. The method of claim 1, wherein the immunogen and PGE2 are administered sequentially.

4. The method of claim 1 wherein the host is a rodent.

5. The method of claim 1 wherein the host is a Balb/c mouse.

6. The method of claim 1 wherein the effective adjuvanting amount of PGE2 is in the range of about 0.1 nmol to about 10 nmol.

7. The method of claim 1 wherein the administration is via a suitable route selected from the group consisting of intraperitoneal, intravenous, subcutaneous, intramuscular, intradermal, or footpad injection.

8. A method for producing antibodies against an immunogen, the method comprising:
administering to a host an immunogen and an effective adjuvanting amount of PGE2 thereby enhancing the immune response against the immunogen, and screening for antibodies, or cells producing antibodies, which are specifically reactive with the immunogen.

9. The method of claim 8, further comprising:
fusing the cells producing antibodies with myeloma cells, and
isolating fused cells which are specifically reactive with the immunogen.

10. The method of claim 8, wherein the immunogen and PGE2 are administered simultaneously.

11. The method of claim 8, wherein the immunogen and PGE2 are administered sequentially.

12. The method of claim 8 wherein the host is a rodent.

13. The method of claim 8 wherein the host is a Balb/c mouse.

14. The method of claim 1 wherein the effective adjuvanting amount of PGE2 is in the range of about 0.1 nmol to about 10 nmol.

15. The method of claim 1 wherein the administration is via a suitable route selected from the group consisting of intraperitoneal, intravenous, subcutaneous, intramuscular, intradermal, or footpad injection.

16. At least one antibody produced by a method according to claim 1.

17. At least one antibody produced by a method according to claim 8.
